(19) United States
(12) Patent Application Publication
(10) Pub. No.: US 2015/0290433 A1
(43) Pub. Date: Oct. 15, 2015

(54) Guidance Device for Mating a Guidewire to a Medical Device

(71) Applicant: C. R. Bard, Inc., Murray Hill, NJ (US)

(72) Inventors: Christian M. Holbert, Salt Lake City, UT (US); Jacob B. Braithwaite, Salt Lake City, UT (US); Richard P. Jenkins, Bluffdale, UT (US)

(21) Appl. No.: 14/678,884

(22) Filed: Apr. 3, 2015

Related U.S. Application Data
(60) Provisional application No. 61/974,939, filed on Apr. 3, 2014.

(51) Int. Cl. A61M 25/09

(52) U.S. Cl. CPC A61M 25/09041; A61M 29/00

(57) ABSTRACT

A guidance device for mating a guidewire with a lumen of a dilator. The guidance device includes a body defining a conduit. The conduit may include a first conduit portion extending from a first end of the body, and may be sized to receive a distal portion of the dilator. The conduit may also include a second conduit portion, extending from a second end of the body, in communication with the first conduit portion. The second conduit portion may be sized to receive the guidewire. The conduit may align the lumen of the dilator with the guidewire when the distal portion of the dilator and the guidewire are received in the conduit such that the guidewire can be inserted into the lumen of the dilator. The body may also include an open slit to enable the guidance device to be removed from the dilator and the guidewire.

### CROSS-REFERENCE TO RELATED APPLICATIONS

This application claims the benefit of U.S. Provisional Patent Application No. 61/974,939, filed Apr. 3, 2014, and titled “Guidance Device for Mating a Guidewire to a Medical Device,” which is incorporated herein by reference in its entirety.

### BRIEF DESCRIPTION OF THE DRAWINGS

A more particular description of the present disclosure will be rendered by reference to specific embodiments thereof that are illustrated in the drawings. It is appreciated that these drawings depict only typical embodiments of the invention and are therefore not to be considered limiting of its scope. Example embodiments of the invention will be described and explained with additional specificity and detail through the use of the accompanying drawings in which:

FIGS. 1A-1D are various views of a guidance device according to one embodiment;

FIG. 2 is a cross-sectional side view of a guidance device in use with a dilator and guidewire according to one embodiment;

FIG. 3 is a perspective view of a guidance device according to one embodiment;

FIGS. 4A-4D are various views of a guidance device according to one embodiment;

FIGS. 5A-5D are various views of a guidance device according to one embodiment;

FIGS. 6A-6D are various views of a guidance device according to one embodiment;

FIGS. 7A-7B are various views of a guidance device according to one embodiment;

FIGS. 8A-8B are various views of a guidance device according to one embodiment;

FIGS. 9A-9B are various views of a guidance device according to one embodiment; and

FIG. 10 is a perspective view of a guidance device according to one embodiment.

### DETAILED DESCRIPTION OF SELECTED EMBODIMENTS

Reference will now be made to figures wherein like structures will be provided with like reference designations. It is understood that the drawings are diagrammatic and schematic representations of exemplary embodiments of the present invention, and are neither limiting nor necessarily drawn to scale.

For clarity it is to be understood that the word “proximal” refers to a direction relatively closer to a clinician using the device to be described herein, while the word “distal” refers to a direction relatively further from the clinician. For example, the end of a catheter placed within the body of a patient is considered a distal end of the catheter, while the catheter end remaining outside the body is a proximal end of the catheter. Also, the words “including,” “has,” and “having,” as used herein, including the claims, shall have the same meaning as the word “comprising.”

Embodiments of the present invention are generally directed to a guidance device for assisting with the loading of a dilator or other suitable medical device on to a guidewire. Dilators and guidewires are commonly employed during insertion procedures to place a catheter within the vasculature or other internal body portion of a patient. The guidance device disclosed herein provides an easy-to-use transition between the relatively small distal opening of the dilator lumen and the guidewire such that the guidewire can be readily received within the lumen of the dilator without undue effort by the clinician placing the catheter. This in turn reduces clinician frustration with what can otherwise be a challenging procedure to mate the guidewire with the dilator, and further reduces the length of the insertion procedure.

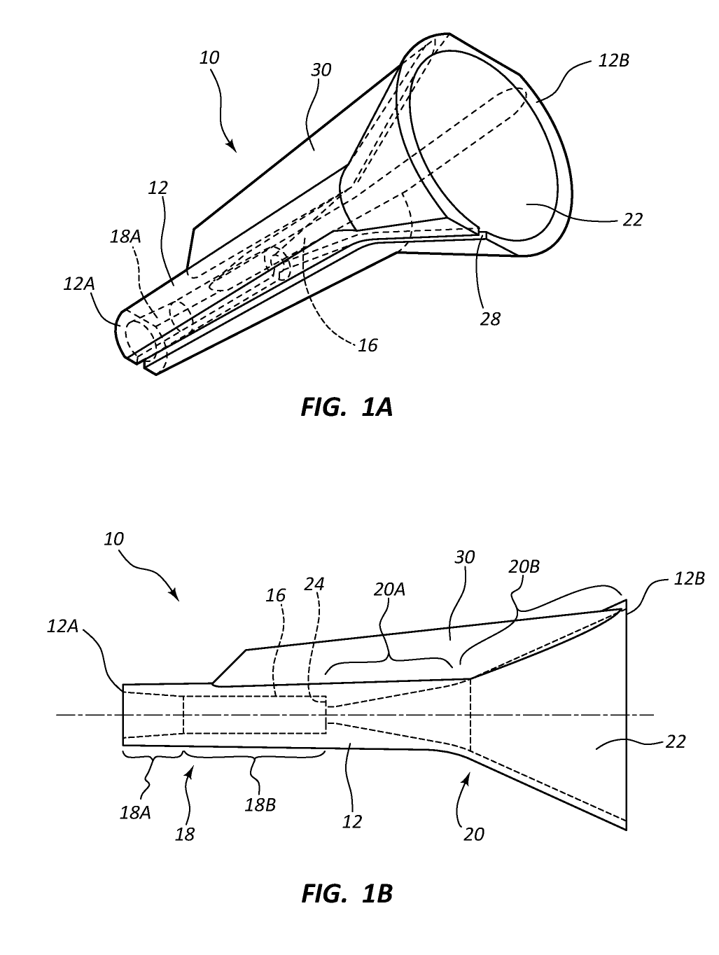

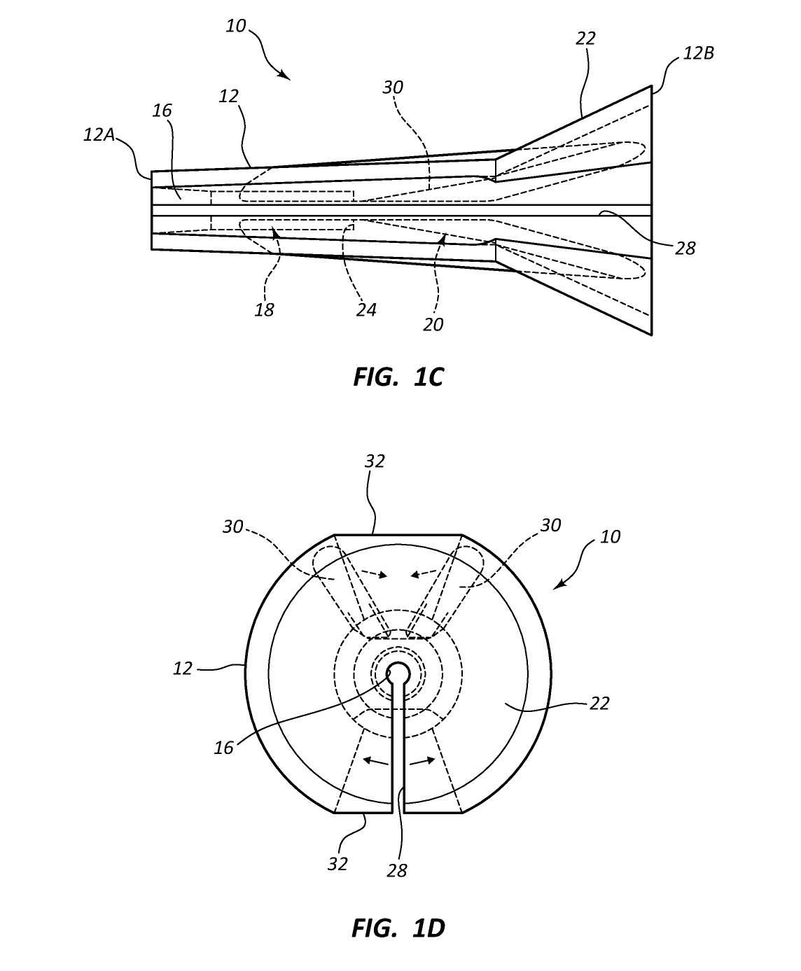

Reference is first made to FIGS. 1A-1D, which depict various views of a guidance device (“device”), generally designated at 10, according to one embodiment. As shown, the guidance device 10 includes an elongate body 12 that extends between a proximal end 12A and a distal end 12B thereof. The device body 12 further defines an axial conduit 16 that extends to and between the proximal and distal ends 12A, 12B, of the body.

In greater detail, the conduit 16 defined by the device body 12 includes a proximal conduit portion 18 extending distally from the proximal end 12A of the body 12 and a distal conduit portion 20 extending proximally from the body distal end 12B so as to communicate with the proximal conduit portion. The proximal conduit portion 18 further includes a tapered portion 18A disposed immediately adjacent the body proximal end 12A and a cylindrical portion 18B immediately adjacent the tapered portion. The tapered portion 18A assists in matching the tapered shaped of the distal portion of a dilator inserted into the proximal conduit portion 18 while both the tapered portion 18A and the cylindrical portion 18B are sized so as to provide a friction fit between the distal portion of the dilator and the proximal conduit portion, thus ensuring acceptable alignment of the dilator lumen with a guidewire.

The distal conduit portion 20 further includes a first tapered portion 20A defining a first taper and a second tapered portion 20B defining a second taper that is less narrow relative the first taper. The second taper portion 20B in the present embodiment is defined by a cone portion 22 of the device body 20. The first and second tapered portions 20A, 20B are sized and configured to enable a clinician to easily insert a guidewire into the distal conduit portion 20 and guide the distal portion of the guidewire into mating alignment with the distal portion of the dilator disposed in the proximal conduit portion 18, as will be seen. In one embodiment, the first tapered portion 20A defines an angle of about 10 degrees, +/−1 degree, with a central longitudinal axis of the of the device body 12, while the second tapered portion 20B defines an angle of about 25 degrees, +/−2 degrees, with the central longitudinal axis. It is appreciated, however, that other angles can be defined by the first, second, and other tapered portions that may be defined by the conduit.

Note that the tapered portions are configured in one embodiment to guide insertion of the guidewire into engagement with the lumen of the dilator smoothly, e.g., without causing piercing of the guidance device by the guidewire or kinking/doubling back of the guidewire. Note that one, two, or more taper regions can be included in the conduit.

FIG. 1B further shows that a shoulder 24 is defined between the meeting point of the tapered portion 18B of the proximal conduit portion 18 and the first tapered portion 20A of the distal conduit portion 20. The shoulder 24 in one embodiment serves as a stop for preventing further advancement of a distal portion of a dilator inserted into the proximal conduit portion 18. In another embodiment, the shoulder is defined to assist in molding the guidance device body. In yet another embodiment, no shoulder is included in the conduit 16. As FIG. 1D shows, the cross sectional shape of the conduit 16 is substantially round, though of varying diameters, as seen in FIGS. 1C and 1D. It is appreciated that the conduit can be configured with different regions, shapes, sizes, etc.

As best seen in FIGS. 1A, 1B, and 1C, a slit 28 is defined longitudinally along the length of the device body 12. The slit 28 enables the dilator and guidewire to be removed from the device 10 after loading of the guidewire into the dilator has been accomplished. The device body 12 in one embodiment includes low density polyethylene, such as LDPE 933i, which renders sufficient pliability to the device 10 so as to enable some deformation of the body such that the dilator and guidewire can be removed via the slit 28. It is appreciated, however, that the device can include one or more of a variety of materials, including other thermoplastics such as polypropylene, TPEs, etc. In one embodiment, the device can include silicone or other suitable thermoset. In one embodiment, the material included in the device includes sufficient strength to hold its form while being sufficiently elastomeric so as to enable limited deformation for removal of the dilator and guidewire therefrom. In one embodiment, the device 10 is manufactured by an injection molding process, though other manufacturing methods may also be employed, including machining, casting, 3-D printing, etc. In one embodiment, the device includes a translucent material to enable the clinician to see into the conduit 16.

FIGS. 1A-1D further show that two tabs 30 are positioned on the device body 12 on an external surface thereof opposite the slit 28. The tabs 30 are configured to be grasped by the fingers of a clinician and pressed toward each other in order to cause the slit 28 to be widened sufficient to enable the dilator distal portion and guidewire to be removed from the conduit 16 after loading of the dilator on to the guidewire. Note that the design, size, configuration, etc., of the tabs can vary from what is explicitly shown and described herein. In the present embodiment, the body 12 of the device further defines longitudinally extending flattened regions 32 that are opposed to one another. The flattened regions 32 assist in deformation of the guidance device body 12 when the tabs 30 are pressed by the clinician so as to ease removal of the dilator distal portion 42 and the guidewire 46 through the slit 28, which further reduces the chance for dilator/guidewire damage.

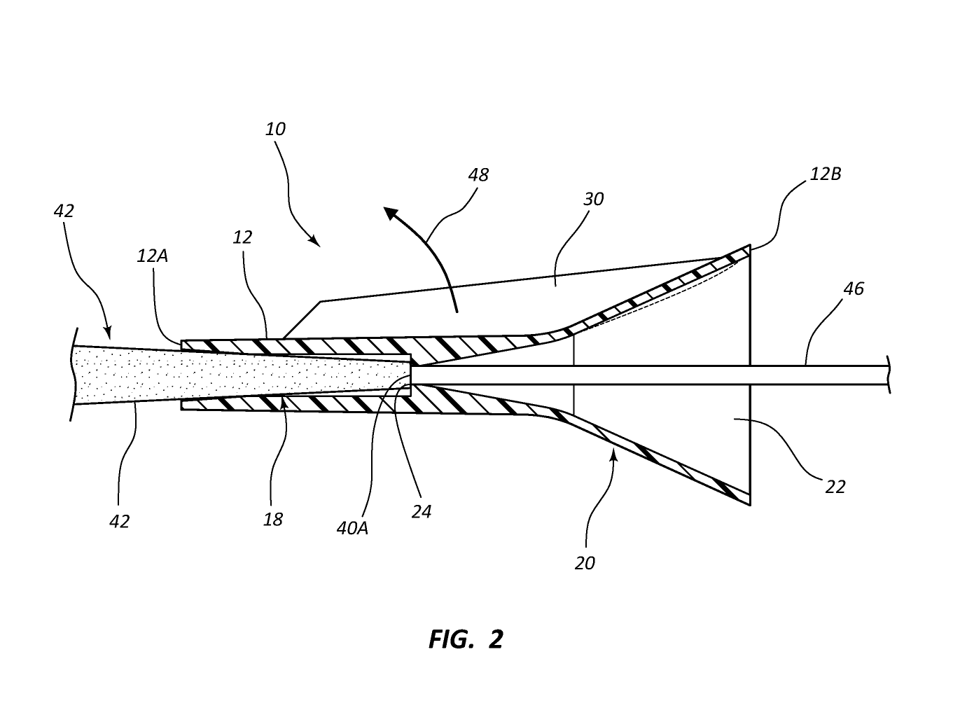

FIG. 2 depicts operation of the device 10 in facilitating the loading of a dilator 40 by a guidewire 46, according to one embodiment. As shown, a distal portion 42 of the dilator 40 is inserted into the proximal conduit portion 18 of the conduit 16 of the guidance device 10 such that a distal tip 40A of the dilator abuts against the shoulder 24 of the conduit. Though shown here as not occupying the entirety of the volume of the proximal conduit portion 18, the distal portion 42 of the dilator 40 in one embodiment entirely fills the conduit volume.

Positioned as described above, the dilator 40 is ready to be loaded with the guidewire 46, a proximal end of which is fed into the distal conduit portion 20 of the conduit 16 via the cone portion 22 of the device body 12. The first and second tapered portions 20A, 20B assist in guiding the proximal end of the guidewire 46 into the conduit 16 and past the shoulder 24 without kinking in order to be inserted into opening of the dilator lumen disposed at the distal tip 40A thereof. Once a sufficient length of the guidewire 46 has been loaded into the lumen of the dilator 40, the clinician can press together the tabs 30 to expand the slit 28 and remove the guidance device 10 from engagement with the guidewire-loaded dilator in the direction indicated by arrow 48.

Note that the dilator/guidewire alignment provided by the guidance device 10 desirably assists with dilator loading onto the guidewire in light of the fact that the distal opening to the dilator lumen and the guidewire are similarly sized. In one embodiment, for instance, the comparison of guidewire outer diameters (first number) to the inner diameter of the dilator lumen (second number) in inches is as follows: 0.032″: 0.035″; 0.035″: 0.037″; 0.036″: 0.040″; 0.038″: 0.040″. The guidance device can be used with dilators and guidewires of a variety of sizes for use with a variety of catheters, including acute CVCs, PICCs, urinary catheters, drainage catheters, hemodialysis catheters, etc. Also, other medical devices can benefit from the principles described herein. In one embodiment, it is appreciated that the guidance device can be pre-loaded on to the distal end of the dilator to simplify its use for the clinician.

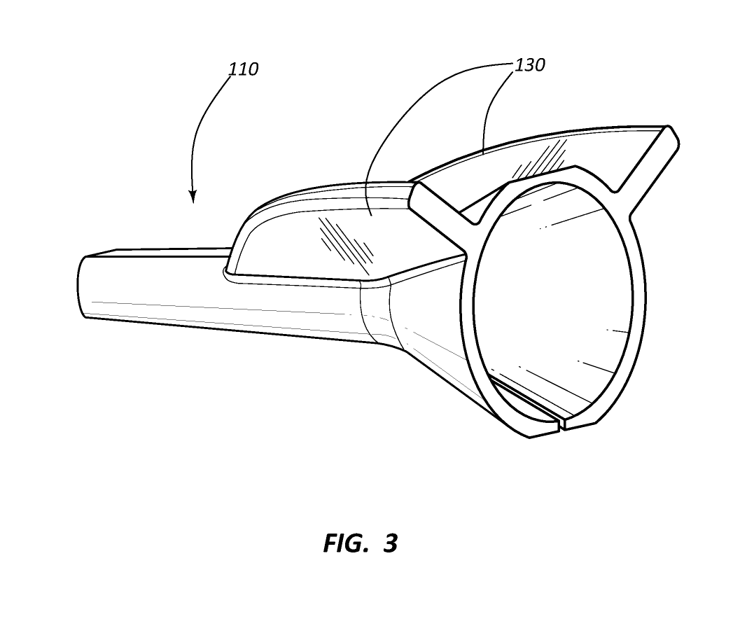

FIG. 3 shows a guidance device 110 according to another embodiment, including one possible configuration of tabs 130. Note that, in another embodiment, the tabs can be placed adjacent to the slit, if desired. Note that these and other variations in the design of the guidance device are therefore contemplated.

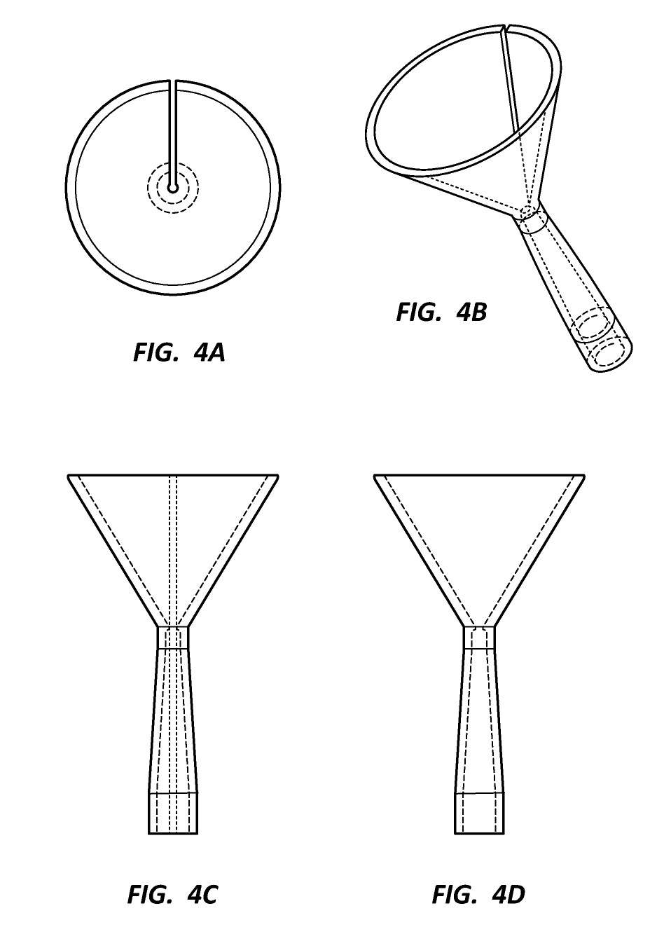

FIGS. 4A-4D depict various views of a guidance device, according to one embodiment. In an embodiment, the guidance device may not include tabs.

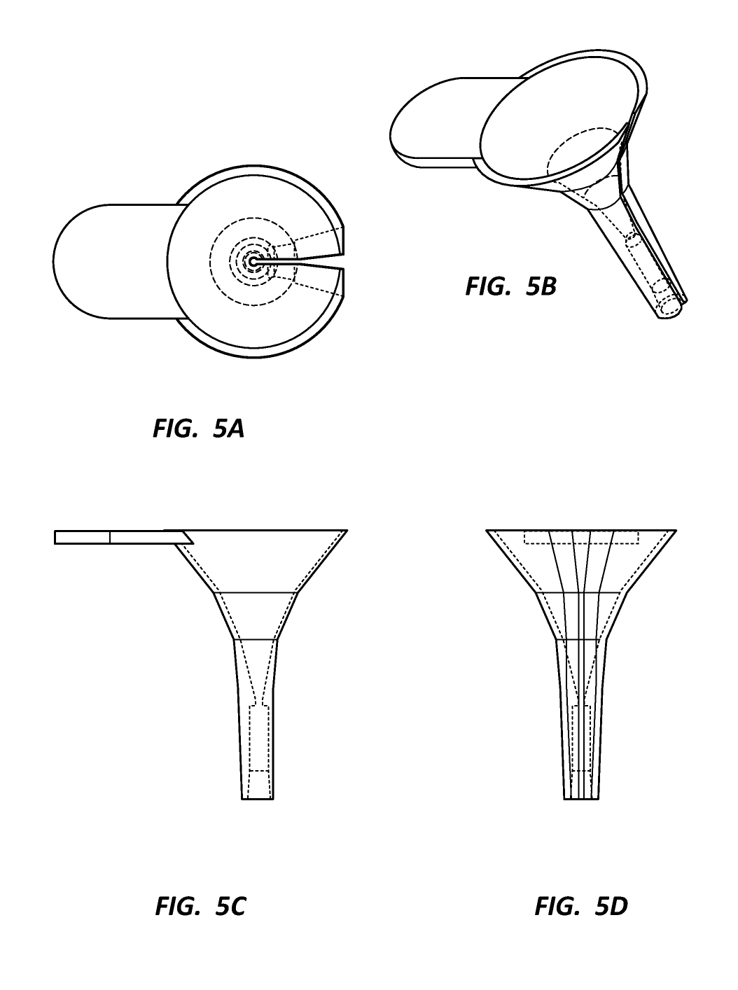

FIGS. 5A-5D depict various views of a guidance device, according to one embodiment. In an embodiment, the tabs can be placed adjacent the distal end, if desired.

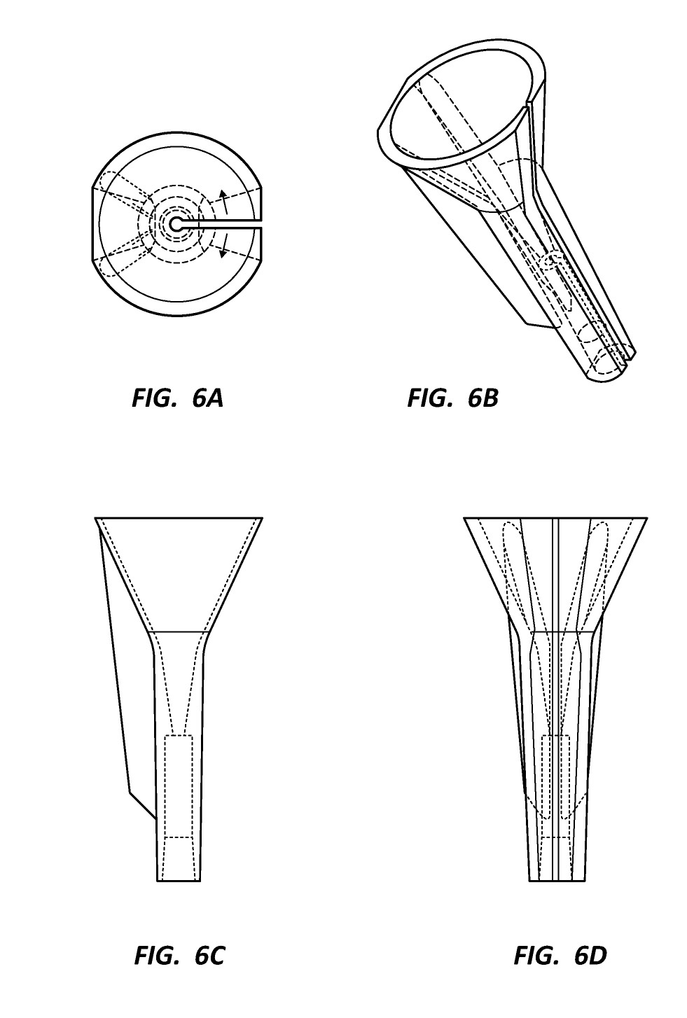

FIGS. 6A-6D depict various views of a guidance device, according to one embodiment.

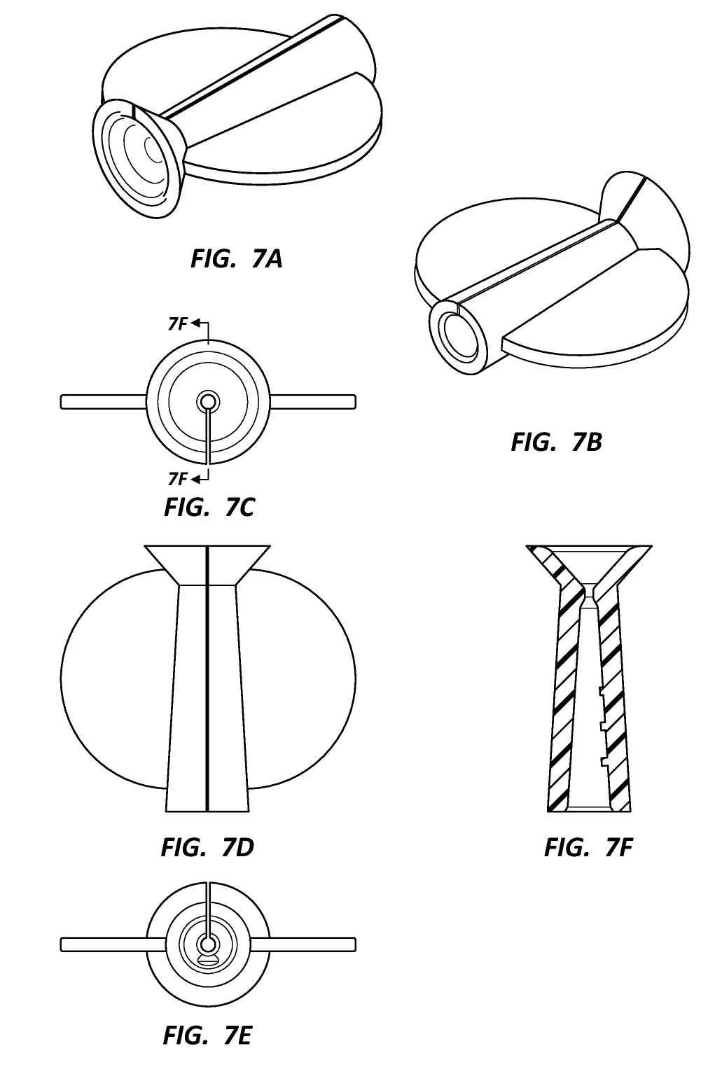

FIGS. 7A-7B depict various views of a guidance device, according to one embodiment.

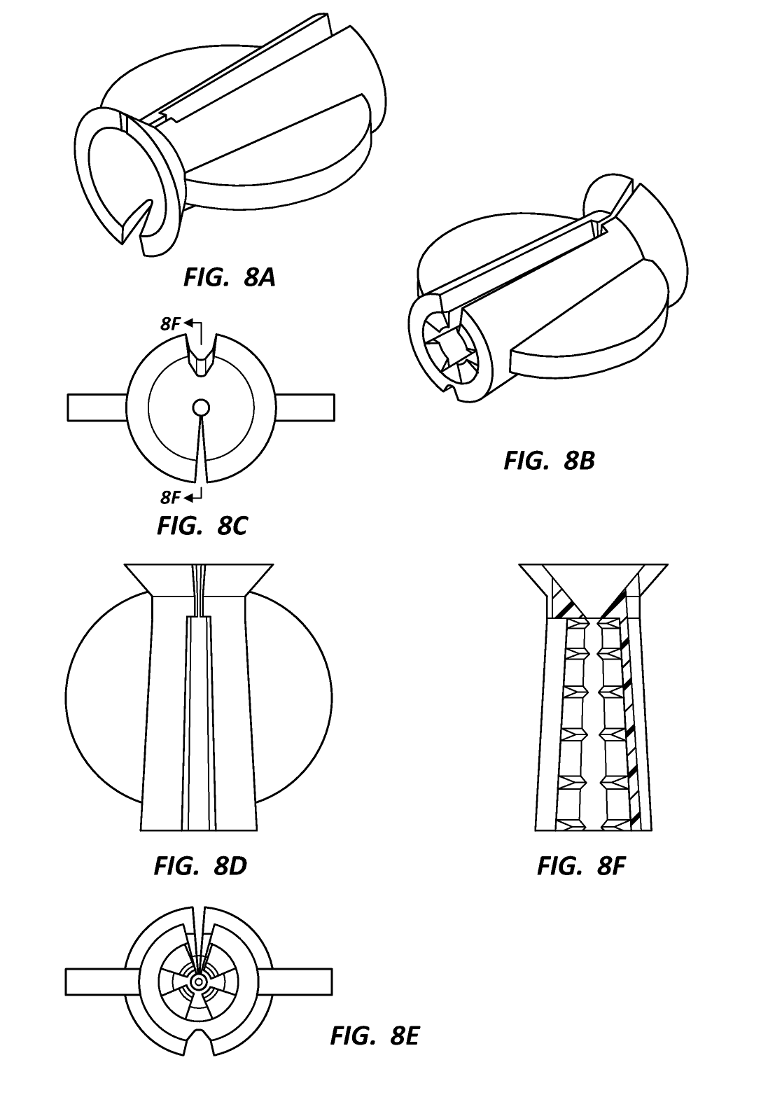

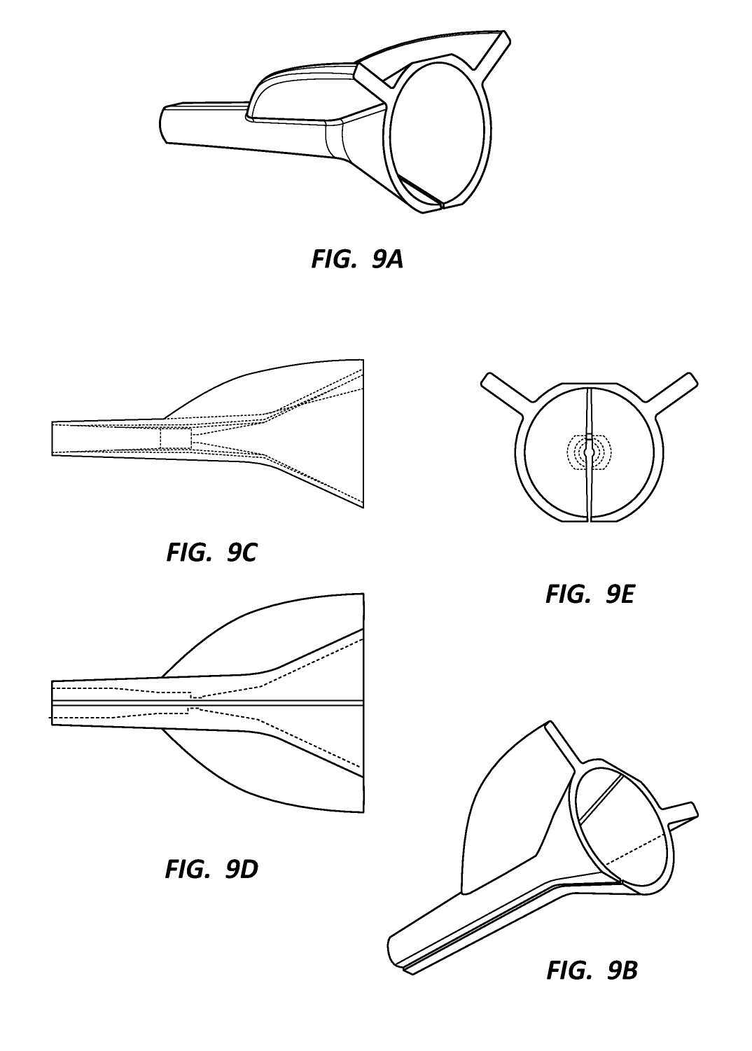

FIGS. 8A-8B depict various views of a guidance device, according to one embodiment.

FIGS. 9A-9B depict various views of a guidance device, according to one embodiment.

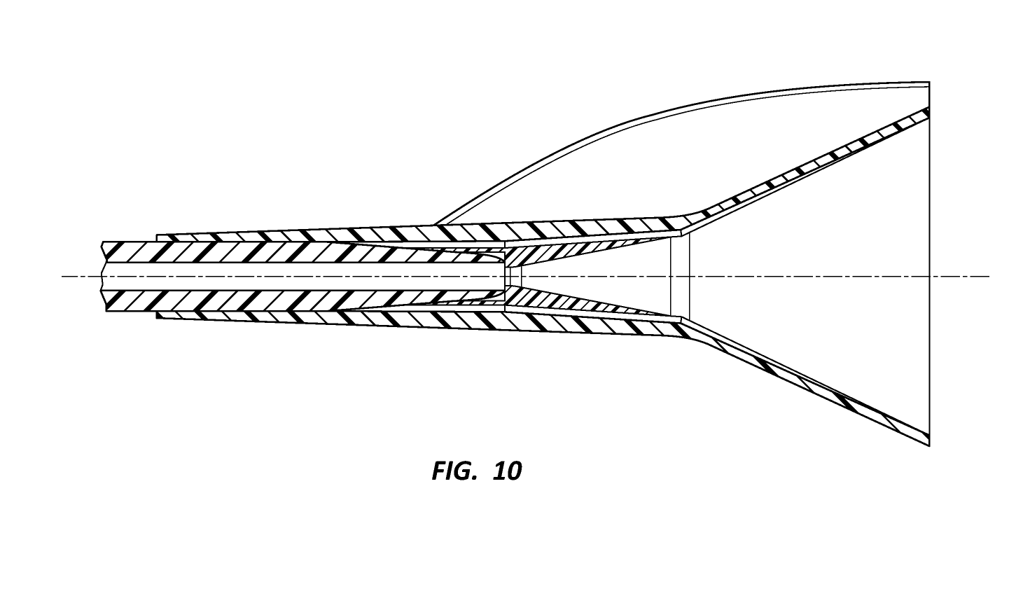

FIG. 10 is a perspective view of a guidance device according to one embodiment.

Embodiments of the invention may be embodied in other specific forms without departing from the spirit of the present disclosure. The described embodiments are to be considered in all respects only as illustrative, not restrictive. The scope of the embodiments is, therefore, indicated by the appended claims rather than by the foregoing description. All changes that come within the meaning and range of equivalency of the claims are to be embraced within their scope.

What is claimed is:

1. A guidance device for mating a guidewire with a lumen of a dilator, comprising:
a body defining a conduit, the conduit including:
a first conduit portion extending from a first end of the body, the first conduit portion sized to receive therein a distal portion of the dilator; and
a second conduit portion extending from a second end of the body the second conduit portion in communication with the first conduit portion, the second conduit portion sized to receive therein the guidewire,
wherein the conduit aligns the lumen of the dilator with the guidewire when the distal portion of the dilator and the guidewire are received in the conduit such that the guidewire can be inserted into the lumen of the dilator; and
an open slit defined in the body to enable the guidance device to be removed from the dilator and the guidewire.

2. The guidance device as defined in claim 1, wherein the conduit further includes a shoulder interposed between the first and second conduit portions.

3. The guidance device as defined in claim 1, further comprising a plurality of tabs included on the body, the tabs being actuatable by a user to open the slit.
